(19) United States
(12) Patent Application Publication
(10) Pub. No.: US 2012/0108812 A1
(43) Pub. Date: May 3, 2012

(54) PROCESS FOR SUBSTITUTED 3-AMINO-5-OXO-4,5-DIHYDRO-[1,2,4]TRIAZINES

(75) Inventors: Hanqing Dong, Syosset, NY (US); Yunyu Mao, Brooklyn, NY (US); Kristen Michelle Mulvhill, Dix Hills, NY (US); Josef A. Rechka, Port Jefferson, NY (US); Douglas S. Werner, Holtsville, NY (US)

(73) Assignee: OSI PHARMACEUTICALS ,INC.

(21) Appl. No.: 13/383,035

(22) PCT Filed: Jul. 8, 2010
(86) PCT No.: PCT/US2010/041278

§ 371 Date: Jan. 9, 2012

Related U.S. Application Data
(60) Provisional application No. 61/224,090, filed on Jul. 9, 2009.

(51) Int. Cl. C07D 253/075

(52) U.S. Cl. 544/182

(57) ABSTRACT

Processes including preparation of trans-4-[(3-amino-5-oxo-4,5-dihydro-[1,2,4]triazin-6-ylmethyl)-carbamoyl]-cyclohexanecarboxylic acid methyl ester, and its conversion to trans-4-(4-amino-5-substituted-imidazo[5,1-f][1,2,4]triazin-7-yl)-cyclohexanecarboxylic acid compounds.

This application claims priority of U.S. Appl. No. 61/224,090 (Jul. 9, 2009), the content of which is incorporated herein by reference in its entirety.

### BACKGROUND OF THE INVENTION

The present invention relates to a process for preparing 3-amino-5-oxo-4,5-dihydro-[1,2,4]triazines and their further conversion to 4-amino-5-substituted-imidazo[5,1-f][1,2,4]triazines.

US 2007/0112005 discloses the preparation of trans-4-[(3-amino-5-oxo-4,5-dihydro-[1,2,4]triazin-6-ylmethyl)-carbamoyl]-cyclohexanecarboxylic acid methyl ester, and its conversion to trans-4-(4-amino-5-substituted-imidazo[5,1-f][1,2,4]triazin-7-yl)-cyclohexanecarboxylic acid methyl ester compounds.

There is a continuing need for alternative and improved processes, including processes having shortened synthetic steps, improved scalability, more efficient isolation and purification of the product, easier handling, better yields, enhanced safety, less reaction time, less consumption of starting materials, reduced environmental contamination, and reduced cost.

### SUMMARY OF THE INVENTION

The present invention relates to a process for the preparation of trans-4-[(3-amino-5-oxo-4,5-dihydro-[1,2,4]triazin-6-ylmethyl)-carbamoyl]-cyclohexanecarboxylic acid methyl ester, and its conversion to trans-4-(4-amino-5-substituted-imidazo[5,1-f][1,2,4]triazin-7-yl)-cyclohexanecarboxylic acid compounds. In one aspect the invention relates to a process for the preparation of trans-4-[(3-amino-5-oxo-4,5-dihydro-[1,2,4]triazin-6-ylmethyl)-carbamoyl]-cyclohexanecarboxylic acid methyl ester. In another aspect the invention relates to a process for the preparation of trans-4-(5-bromo-4-oxo-3,4-dihydro-imidazo[5,1-f][1,2,4]triazin-7-yl)-cyclohexanecarboxylic acid methyl ester. Another aspect of the invention relates to a process for the preparation of trans-4-(4-amino-5-substituted-imidazo[5,1-f][1,2,4]triazin-7-yl)-cyclohexanecarboxylic acid compounds wherein said 5-substituted group is an optionally substituted aryl or a heteroaryl group. In another aspect the invention relates to a process for the preparation of trans-4-[4-amino-5-(7-methoxy-1H-indol-2-yl)-imidazo[5,1-f][1,2,4]triazin-7-yl]-cyclohexanecarboxylic acid or a pharmaceutically acceptable salt thereof, such as, for example, trans-4-[4-amino-5-(7-methoxy-1H-indol-2-yl)-imidazo[5,1-f][1,2,4]triazin-7-yl]-cyclohexanecarboxylate 2-hydroxy-1,1-bis-hydroxymethyl-ethyl-ammonium.

### DETAILED DESCRIPTION OF THE INVENTION

In one aspect the invention relates to a process for the preparation of a compound of formula (I) or a salt thereof:

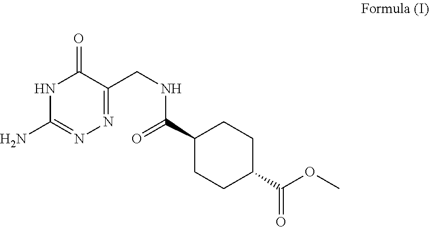

said process comprising a reaction step according to Scheme 1:

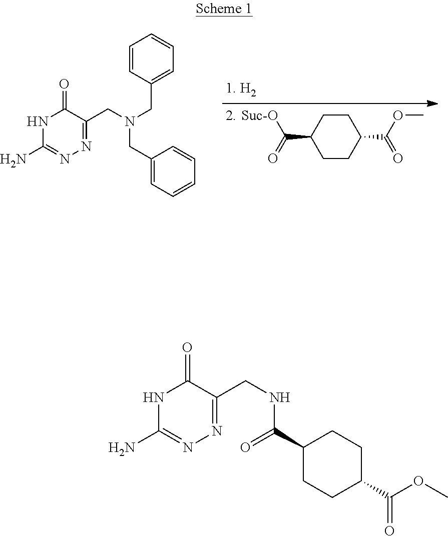

in which 3-amino-6-((dibenzylamino)methyl)-1,2,4-triazin-5(4H)-one is first hydrogenated in the presence of a suitable catalyst, a suitable acid, a suitable solvent and at a suitable reaction temperature. Suitable catalysts include, but are not limited to, 10% palladium on carbon. Suitable acids include, but are not limited to, acetic acid. Suitable solvents include, but are not limited to, water or acetic acid and mixtures thereof. Suitable reaction temperatures can be about 30° C. to 90° C.

Upon removal of the catalyst, such as by filtration, the resulting intermediate product, preferably still in solution, is reacted with trans-4-[(2,5-dioxopyrrolidin-1-yl)oxy]carbonylmethylcyclohexanecarboxylate in the presence of a suitable base to provide a compound of formula (I). Suitable bases include, but are not limited to, triethylamine or sodium carbonate. The reaction can be carried out at about atmospheric pressure although higher or lower pressures are used if desired. Substantially equimolar amounts of reactants can be used.

Said compound of Formula (I) can be obtained according to Scheme 1 on a scale of at least about 1.5 kg, at least about 5 kg, or at least about 10 kg in a yield of at least about 60%, 70%, 80%, or 90%, without requiring purification.

In another aspect, the invention relates to a process of the preparation of a compound of formula (II) or a salt thereof, wherein the compound of formula (I) is further reacted according to reaction Scheme 2:

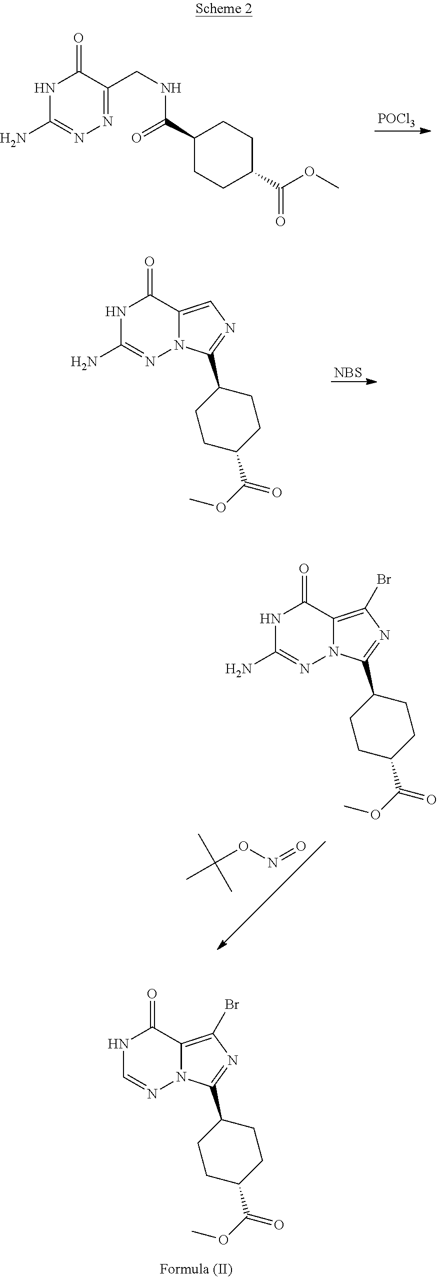

wherein:

Step (A): the compound of formula (I) is reacted with a suitable amount of POCl₃ in a suitable solvent, preferably under reflux, to provide trans-4-(2-amino-4-oxo-3,4-dihydro-imidazo[5,1-f][1,2,4]triazin-7-yl)-cyclohexanecarboxylic acid methyl ester.

Is some embodiments, about 1.3 to 2.0 equivalents POCl₃ compared to the amount of the compound of Formula (I) can be used. Suitable solvents include, but are not limited to, acetonitrile, 1,2-diethoxyethane, dimethoxyethane, or mixtures.

The product from said step (A) can be obtained on a scale of at least about 1.5 kilogram without purification, and the yield of said step (A) can be at least about 95% at a scale of at least about 1.5 kilogram of the product.

Step (B): the product from step (A) is reacted with N-bromosuccinimide in a suitable solvent at a suitable reaction temperature for a suitable reaction time to provide trans-4-(2-amino-5-bromo-4-oxo-3,4-dihydro-imidazo[5,1-f][1,2,4]triazin-7-yl)-cyclohexanecarboxylic acid methyl ester.

Suitable reaction temperatures are about −10° C. to 40° C. Suitable solvents include, but are not limited to dimethylformamide, acetone, ethyl acetate, acetonitrile, tetrahydrofuran, or mixtures. Suitable reaction times can be about 10 to 30 min after the addition of N-bromosuccinimide is complete.

The product from said step (B) can be obtained on a scale of at least about 2 kg, at a yield of said step (B) of at least about 79% or at least about 95% without requiring purification.

Step (C): the product from step (B) is reacted with a suitable amount of tert-butyl nitrite in a suitable solvent to provide a compound of formula (II).

Suitable solvents include, but are not limited to, tetrahydrofuran, dimethylformamide, or mixtures. Suitable amounts of tert-butyl nitrite can be about 2 to 3 equivalents compared to the amount of product from step (B).

The product from said step (C) can be obtained on a scale of at least about 2 kg in a yield of said step (C) of at least about 80% without requiring purification.

The reactions according to Scheme 2 can be carried out at about atmospheric pressure although higher or lower pressures can be used. The final product can be obtained on a scale of at least about 2 kg without requiring purification, and the overall yield of Scheme 2 can be at least about 60%, 70%, 75%, or higher

In another aspect, the invention relates to a process of the preparation of a compound of Formula (III) or a salt thereof, wherein the compound of Formula (II) is further reacted according to reaction Scheme 3:

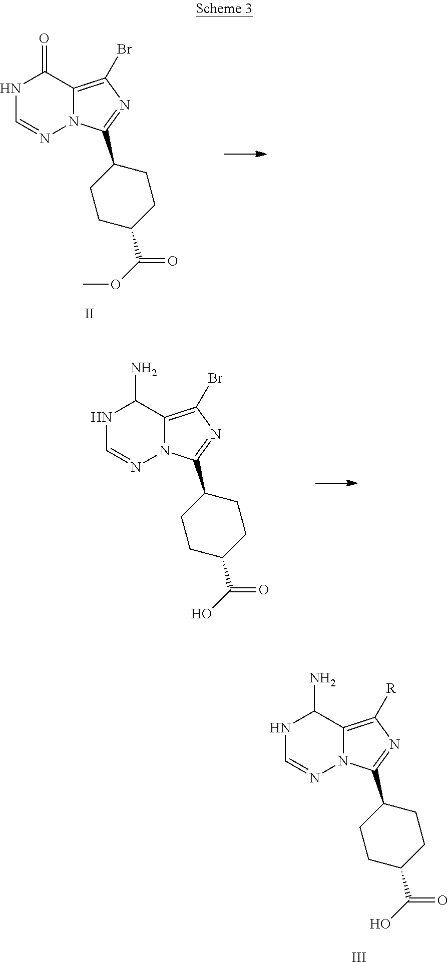

wherein:

Step (D): the compound of Formula (II) is first reacted with 1,2,4-triazole and POCl₃, in the presence of base such as pyridine or triethylamine in acetonitrile, then the resulting intermediate compound is reacted with ammonia, then hydrolyzed with a suitable base to provide trans-4-(4-amino-5-bromo-imidazo[5,1-f][1,2,4]triazin-7-yl)-cyclohexanecarboxylic acid.

The ammonia may be in a suitable alcohol solvent, including, but not limited to, ethanol or isopropanol. Suitable bases include, but are not limited to, NaOH.

The step (D) product can be obtained on a scale of at least about 1.25 kilogram at a yield of said step (D) of about 90% or at least about 95% without requiring purification.

Step (E): the product from step (D) is reacted with R-boronic acid/ester in the presence of a suitable ligand, a suitable catalyst and a suitable base, and in a suitable solvent to provide a compound of formula (III); wherein R is an optionally substituted aryl or a heteroaryl group.

The aryl group can be selected from the group consisting of phenyl, 4-chlorophenyl, 4-fluorophenyl, 4-bromophenyl, 3-nitrophenyl, 2-methoxyphenyl, 2-methylphenyl, 3-methyphenyl, 4-methylphenyl, 4-ethylphenyl, 2-methyl-3-methoxyphenyl, 2,4-dibromophenyl, 3,5-difluorophenyl, 3,5-dimethylphenyl, 2,4,6-trichlorophenyl, 4-methoxyphenyl, naphthyl, 2-chloronaphthyl, 2,4-dimethoxyphenyl, 4-(trifluoromethyl)phenyl, and 2-iodo-4-methylphenyl. The heteroaryl group can be selected from the group consisting of 2-, 3- or 4-pyridinyl, pyrazinyl, 2-, 4-, or 5-pyrimidinyl, pyridazinyl, triazolyl, tetrazolyl, imidazolyl, 2- or 3-thienyl, 2- or 3-furyl, pyrrolyl, oxazolyl, isoxazolyl, thiazolyl, isothiazolyl, oxadiazolyl, thiadiazolyl, quinolyl, isoquinolyl, benzimidazolyl, benzotriazolyl, benzofuranyl, indole and benzothienyl. Said aryl or heteroaryl group can be further substituted with one or more independent substituents selected from the group consisting of C₁-C₁₀ alkyl, halo, cyano, hydroxy, C₁-C₁₀ alkoxy and phenyl.

Regarding step (E), said suitable ligand includes, but are not limited to, 3,3′,3″-phosphinidynetris(benzenesulfonic acid) trisodium salt, or 4,4′-(phenylphosphinidene)bis-benzensulfonic acid, dipotassium salt hydrate. Said suitable catalyst includes, but are not limited to, palladium (II) acetate. Said suitable solvent includes, but are not limited to, a mixture of water, ethanol, and tetrahydrofuran. Said suitable base includes, but not limited to, Na₂CO₃.

The product from said step (E) can be obtained on a scale of at least about 1 kg at a yield of said step (E) of at least about 80% without requiring purification.

The above-described reactions according to Scheme 3 can be carried out at about atmospheric pressure although higher or lower pressures can be used.

According to the Scheme 3 process, the final product can be obtained on a scale of at least about 1 kg or at least about 5 kg or 10 kg, with an overall yield of least about 72% without requiring purification.

In an aspect of step (E) according the above-described scheme 3, said step (E) is conducted according to reaction Scheme 4:

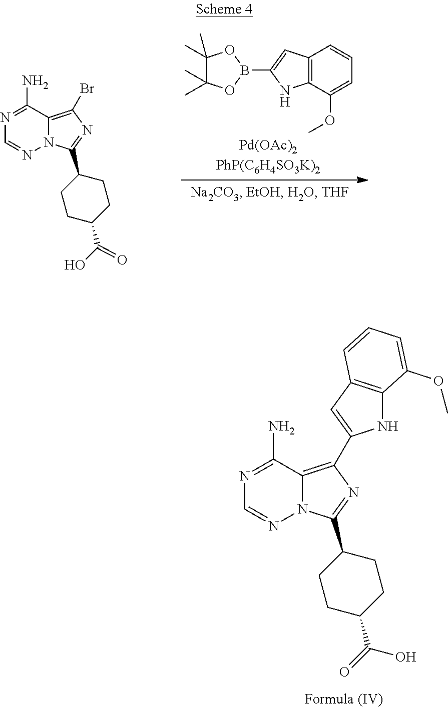

wherein trans-4-(4-amino-5-bromo-imidazo[5,1-f][1,2,4]triazin-7-yl)-cyclohexanecarboxylic acid is reacted with 7-methoxy-2-(4,4,5,5-tetramethyl[1,3,2]-dioxaborolan-2-yl)-1H-indole to provide a compound of formula (IV).

In a typical process of preparation of the compound of Formula (IV) according to Scheme 4, the product from said process can be obtained on a scale of at least about 1 kg, 5 kg, or 10 kg, at a yield of at least about 80% without requiring purification.

In another aspect, the present invention is related to a process of preparation of a compound of Formula (V), wherein the compound of Formula (IV) is further reacted according to reaction Scheme 5:

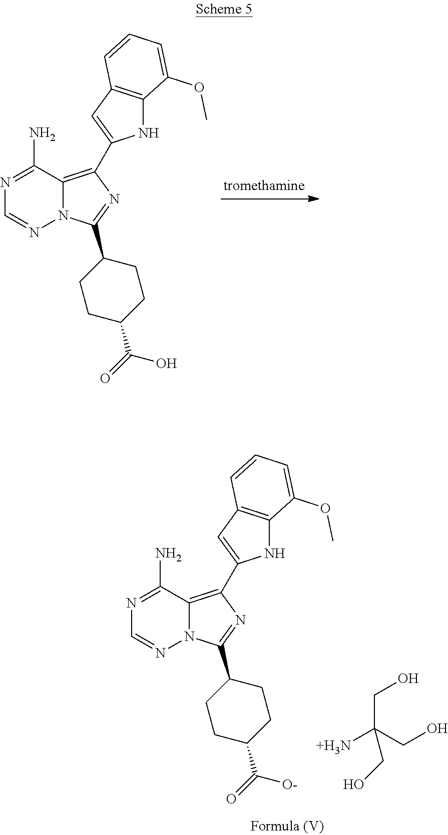

wherein the compound of Formula (IV) is reacted with tris(hydroxymethyl)aminomethane in ethanol and water to provide a compound of Formula (V).

In a typical process of preparation of the compound of Formula (V), said process is conducted under reflux or a slurry at about 40° C. The product of said process can be obtained on a scale of at least about 1.5 kg in a yield at least about 95% without requiring purification.

All processes of preparation, as described above, are supplemented by synthetic methods known in the art of organic chemistry, or modifications and derivatizations that are familiar to those of ordinary skill in the art. The starting materials used herein are commercially available or may be prepared by routine methods known in the art (such as those methods disclosed in standard reference books such as the Compendium of Organic Synthetic Methods, Vol. I-VI (published by Wiley-Interscience)).

During any of the above and/or following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; T. W. Greene and P. G. M. Wuts, Protective Groups in Organic Chemistry, John Wiley & Sons, 1991, and T. W. Greene and P. G. M. Wuts, Protective Groups in Organic Chemistry, John Wiley & Sons, 1999, which are hereby incorporated by reference.

### EXAMPLES

### Example 1

### 3-Amino-6-[(dibenzylamino)-methyl]-4H-[1,2,4]triazin-5-one (A)

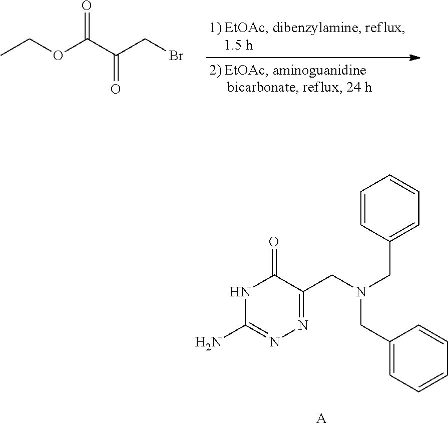

To a 20 L jacketed reactor equipped with a mechanical stirrer, reflux condenser and thermometer was added dibenzylamine (3754 g, 18.46 mol) and EtOAc (9 L). The clear solution was heated to 60° C. Ethyl bromopyruvate (2.000 kg, 9.230 mol) was added over 23 min. The reaction mixture started to turn yellow with a white precipitate forming (dibenzylamine hydrobromide) as it began to exotherm to reflux. The jacket temperature was maintained at 60° C. After addition was complete, the reaction was heated at reflux (80° C.) for 1.5 h. The reaction was cooled to 50° C. and the precipitate was removed by filtration and washed with EtOAc (2×1 L). The dark solution was concentrated in vacuo to an oil/slurry. The slurry was dissolved in EtOH (15 L) and added to a 20 L jacketed reactor equipped with a mechanical stirrer, reflux condenser and thermometer containing aminoguanidine bicarbonate (1295 g, 9.230 mol). The reaction was heated at reflux for 24 h. The reaction was cooled to 50° C. and the brown precipitate was isolated by filtration. The precipitate was washed with water (3 L) and EtOH (2 L). The precipitate was dried at 70° C. under high vacuum to afford about 1.5 kilogram of 3-amino-6-[(dibenzylamino)-methyl]-4H-[1,2,4]triazin-5-one. The yield was about 50%. ¹H NMR (400 MHz, DMSO-d₆): δ 3.47 (s, 1H), 3.63 (s, 1H), 6.68 (br s, 1H), 6.68 (br s, 1H), 7.19-7.23 (m, 2H), 7.27-7.32 (m, 4H), 7.35-7.38 (m, 4H).

### Example 2

### trans-4-[(2,5-dioxopyrrolidin-1-yl)oxy]carbonylmethylcyclohexanecarboxylate

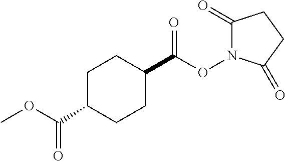

To a 5 L reactor was added N-hydroxysuccinimide (132.9 g, 1.155 mol), trans-4(methoxycarbonyl)cyclohexanecarboxylic acid (195.5 g, 1.050 mol), and DCM (2.0 L). N-(3-Dimethylaminopropyl)-N′-ethylcarbodiimide hydrochloride (241.5 g, 1.260 mol) was added over 10 minutes (temp. at start of addition was 15° C. and after addition, the reaction slowly exothermed to 26° C.). Reaction went from a cloudy solution to a suspension upon start of EDC addition to a clear solution after addition was complete. After 1.5 h, the reaction was washed with H₂O (3×650 mL), dried over MgSO₄, filtered, concentrated in vacuo and the solvent was exchanged for hexanes. The resultant suspension was filtered and dried under high vacuum at room temperature, and trans-4-[(2,5-dioxopyrrolidin-1-yl)oxy]carbonylmethylcyclohexanecarboxylate was obtained as a white solid (293.2 g, 98.6% yield). ¹H NMR (400 MHz, CDCl₃): δ 1.51 (dddd, 2H, J=12.8, 12.8, 12.8, 2.8 Hz), 1.62 (dddd, 2H, J=12.8, 12.8, 12.8, 2.8 Hz), 2.08-2.14 (m, 2H), 2.20-2.24 (m, 2H), 2.34 (tt, 1H, J=11.6, 3.6 Hz), 2.64 (tt, 1H, J=11.6, 3.6 Hz), 2.83 (br s, 4H), 3.68 (s, 3H).

### Example 3

### trans-4-[(3-amino-5-oxo-4,5-dihydro-[1,2,4]triazin-6-ylmethyl)-carbamoyl]-cyclohexanecarboxylic acid methyl ester

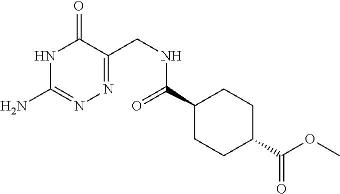

Into a 20 L jacketed reaction vessel equipped with a sparge tube and thermocouple was carefully added 3-amino-6-[(dibenzylamino)-methyl]-4H-[1,2,4]triazin-5-one (2.50 kg, 7.78 mol) to AcOH (1.19 L, 21.0 mol) at a rate to control off-gassing. H₂O (12.5 L) was then added followed by 10% Pd/C (229 g, 61.7% water, 0.0824 mol). The reactor was vacuum purged and backfilled with nitrogen (repeated 2×) and then vacuum purged and backfilled with hydrogen gas (repeated 2×). The reaction was heated at 55° C. while hydrogen gas was added to the reaction through the sparge tube. Once the reaction was complete by HPLC, it was cooled to rt and filtered through a plug of Celite. The filtrate was transferred to a clean 20 L jacketed reactor and charged with triethylamine (3.04 L, 21.8 mol) and MeCN (2.5 L). The reaction was heated to 50° C. and trans-4-[(2,5-dioxopyrrolidin-1-yl)oxy]carbonylmethylcyclohexanecarboxylate (2.20 kg, 7.78 mol) was added. Upon reaction completion, the reaction was cooled to rt and filtered. The solid was washed with water (5.0 L) and MeCN (5.0 L) and dried in vacuo at 40-50° C. to yield the title compound as an off-white solid (1.52 kg, 63% 2-step yield). ¹H NMR (400 MHz, DMSO-d₆): δ1.25-1.42 (m, 4H), 1.75-1.78 (m, 2H), 1.88-1.93 (m, 2H), 2.14 (tt, 1H, J=11.6, 3.6 Hz), 2.27 (tt, 1H, J=11.2, 3.2 Hz), 3.58 (s, 3H), 4.04 (d, 2H, J=5.2 Hz), 6.75 (br s, 2H), 7.85 (dd, 1H, J=5.6, 5.6 Hz), 11.92 (br s, 1H).

### Example 4

### The Preparation of trans-4-(2-amino-4-oxo-3,4-dihydro-imidazo[5,1-j][1,2,4]triazin-7-yl)-cyclohexanecarboxylic acid methyl ester

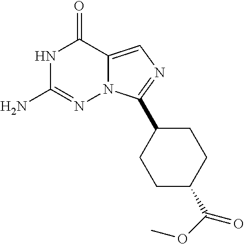

trans-4-[(3-Amino-5-oxo-4,5-dihydro-[1,2,4]triazin-6-ylmethyl)-carbamoyl]-cyclohexanecarboxylic acid methyl ester (1.65 kg, 5.31 mol) and MeCN (5.8 L) were added into a 20 L jacketed reactor equipped with an addition funnel, condenser, and nitrogen inlet. POCl₃ (1.05 L, 11.1 mol) was then added to the thick suspension within 20 min and the reaction was heated to reflux. Once complete by LCMS, the reaction was cooled to 0° C. Potassium carbonate (3.08 kg, 22.3 mol) in water (6.0 L) was added to the reaction until pH 8 at a rate to keep the temperature at <30° C. (1 h). Once the quench was complete, the suspension was stirred at rt for 20 min and then cooled to 6° C. The suspension was filtered and the light brown solid was washed with water (7.0 L). The solid was dried in vacuo at 40° C. overnight to provide the title compound (1.47 kg, 95% yield). ¹H NMR (400 MHz, DMSO-d₆): δ1.43 (dddd, 2H, J=13.2, 13.2, 13.2, 3.2 Hz), 1.61 (dddd, 2H, J=12.8, 12.8, 12.8, 2.8 Hz), 1.93-2.03 (m, 4H), 2.38 (tt, 1H, J=11.6, 3.6 Hz), 2.98 (tt, 1H, J=12.0, 3.6 Hz), 3.61 (s, 3H), 6.23 (br s, 2H), 7.46 (s, 1H), 11.06 (br s, 1H).

### Example 5

### trans-4-(2-Amino-5-bromo-4-oxo-3,4-dihydro-imidazo[5,1-f][1,2,4]triazin-7-yl)-cyclohexanecarboxylic acid methyl ester

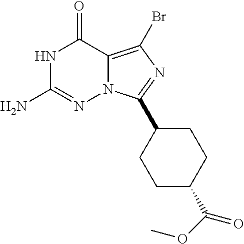

trans-4-(2-Amino-4-oxo-3,4-dihydro-imidazo[5,1-f][1,2,4]triazin-7-yl)-cyclohexanecarboxylic acid methyl ester (2.05 kg, 7.04 mol) was suspended in DMF (10.1 L) in a 20 L jacketed reactor. Solid NBS (1.46 kg, 8.21 mol) was added over 10 min and the reaction exothermed from 21° C. to 37° C. (jacket set at −15° C.). The reaction was then stirred at rt. After 1.5 h, a sample was taken and HPLC showed complete conversion. H₂O (11.4 L) was added in the following way: The reactor jacket was set at 10° C. and water was added until crystallization commenced (6 L), addition was ceased and the suspension was stirred for 10 min and then the remaining water was added in one portion. The suspension was filtered and the solid was washed with water (4 L). The brick red solid was dried in vacuo at 50° C. to provide the title compound (2.05 kg, 79% yield). ¹H NMR (400 MHz, DMSO-d₆): δ 1.40 (dddd, 2H, J=12.8, 12.8, 12.8, 3.2 Hz), 1.57 (dddd, 2H, J=12.8, 12.8, 12.8, 3.2 Hz), 1.91-2.02 (m, 4H), 2.38 (tt, 1H, J=12.0, 3.2 Hz), 2.97 (tt, 1H, J=12.0, 3.6 Hz), 3.60 (s, 3H), 6.20 (s, 2H), 10.85 (s, 1H).

### Example 6

### trans-4-(5-Bromo-4-oxo-3,4-dihydro-imidazo[5,1-f][1,2,4]triazin-7-yl)-cyclohexanecarboxylic acid methyl ester

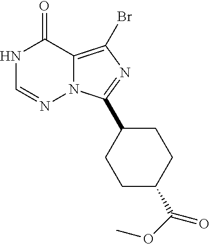

A 50 L three-necked round-bottomed flask equipped with a mechanical stirrer, thermocouple, nitrogen inlet, addition funnel, drying tube and cooling bath was charged with methyl trans-4-(2-amino-5-bromo-4-oxo-3,4-dihydro-imidazo[5,1-f][1,2,4]triazin-7-yl)-cyclohexanecarboxylate (1.99 kg, 5.40 mol) and THF (36 L). The reaction was stirred to give a tan slurry and the cooling bath was filled with cold tap water. After 30 min, the reaction was slowly charged with tert-butyl nitrite (1.43 L, 10.8 mol) over 90 min maintaining the temperature of the reaction mixture at 15-25° C. The reaction was stirred at ambient temperature over a minimum of 18 hours in which a light brown clear solution formed. The reaction was monitored by HPLC until it was complete. Charcoal (120 g) was added portionwise to the reaction mixture and the reaction was filtered through a 2-3 inch celite bed. The reaction mixture was concentrated under reduced pressure at 25-30° C. to approximately 6-7 L of total volume (the product precipitated during the concentration of THF resulting in a thick yellow slurry). Once the total volume was approximately 6 L, heptane (6 L) was added to precipitate more of the product. The slurry was stirred at 0-−10° C. for a minimum of 2 h. The reaction was filtered and the solid was washed with MTBE (3×3 L). The solids were dried in vacuo at rt to provide trans-4-(5-bromo-4-oxo-3,4-dihydro-imidazo[5,1-f][1,2,4]triazin-7-yl)-cyclohexanecarboxylic acid methyl ester (1.57 kg, 82% yield). ¹H NMR (400 MHz, DMSO-d₆): δ 1.46 (dddd, 2H, J=12.4, 12.4, 12.4, 2.8 Hz), 1.61 (dddd, 2H, J=12.8, 12.8, 12.8, 3.2 Hz), 1.95-2.03 (m, 4H), 2.40 (tt, 1H, J=12.0, 3.6 Hz), 3.08 (tt, 1H, J=12.0, 3.2 Hz), 3.61 (s, 3H), 7.90 (d, 1H, J=4.0 Hz), 10.85 (d, 1H, J=3.6 Hz).

### Example 7

### trans-4-(4-Amino-5-bromo-imidazo[5,1-f][1,2,4]triazin-7-yl)-cyclohexanecarboxylic acid

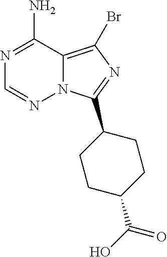

A 50 L three-necked round-bottomed flask equipped with a mechanical stirrer, thermocouple, nitrogen inlet, addition funnel, drying tube and cooling bath was charged with 1,2,4-triazole (1.54 kg, 22.3 mol) and pyridine (6.5 L). The slurry was cooled to 10-15° C. and charged with phosphorous oxychloride (679 mL, 7.42 mol) while maintaining the reaction temperature at <40° C. The suspension was stirred for 20 min. trans-4-(5-Bromo-4-oxo-3,4-dihydro-imidazo[5,1-f][1,2,4]triazin-7-yl)-cyclohexanecarboxylic acid methyl ester (1.32 kg, 3.71 mol) was dissolved in pyridine (6.5 L) and the cloudy solution was charged to the reaction over 30 min while maintaining the reaction temperature at 25-30° C. The reaction was stirred at rt until complete (˜18 h) and then cooled to −15° C. The reaction was then charged with 8M ammonia in ethanol (7.00 L, 55.7 mol) over 45 min while maintaining the reaction temperature at <0° C. The reaction was then stirred for 1 h without cooling. Upon reaction completion, the reaction was concentrated in vacuo. The residue was then slurried in ethanol (13 L) and cooled to 10° C. A solution of NaOH (50% aq. w/w, 3.36 L, 63.1 mol) diluted with water (10 L) was charged to the reaction over 2-3 h while maintaining the reaction mixture at 10-20° C. The reaction was stirred at rt until complete and citric acid (7.13 kg, 37.1 mol) dissolved in water (7.0 L) was added to the reaction at a rate maintaining the reaction temperature at ≦20° C. The suspension was then stirred at rt for 12 h and then filtered. The tan solid was washed with water (3×2.6 L) and ethanol (3×2.6 L) to provide trans-4-(4-amino-5-bromo-imidazo[5,1-j][1,2,4]triazin-7-yl)-cyclohexanecarboxylic acid (1265 g, 99% yield). ¹H NMR (400 MHz, DMSO-d₆): δ 1.44 (dddd, 2H, J=12.4, 12.4, 12.4, 2.8 Hz), 1.60 (dddd, 2H, J=14.0, 14.0, 14.0, 4.0 Hz), 1.95-2.02 (m, 4H), 2.27 (tt, 1H, J=12.0, 3.2 Hz), 3.12 (tt, 1H, J=12.0, 3.6 Hz), 7.06 (br s, 1H), 7.86 (s, 1H), 8.48 (br s, 1H), 11.2 (br s, 1H).

### Example 8

### trans-4-[4-Amino-5-(7-methoxy-1H-indol-2-yl)-imidazo[5,1-f][1,2,4]triazin-7-yl]-cyclohexanecarboxylic acid

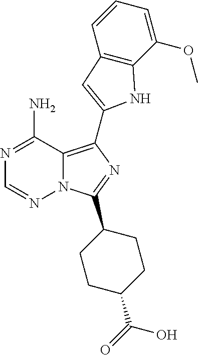

trans-4-(4-Amino-5-bromo-imidazo[5,1-f][1,2,4]triazin-7-yl)-cyclohexanecarboxylic acid (1.10 kg, 3.22 mol) was suspended in water (11 L) and nitrogen gas was bubbled through the mixture. Sodium carbonate (1.02 kg, 9.66 mol) was added and the mixture was stirred at rt for 10 min. The mixture was diluted with ethanol (11 L) and stirring was continued for 0.5 h while the system was degassed with nitrogen. 4,4′-(Phenylphosphinidene)bis-benzensulfonic acid, dipotassium salt hydrate (8.00 g, 16.1 mmol) and palladium (II) acetate (2.00 g, 8.00 mmol) were added and the mixture was then heated to 60° C. 7-Methoxy-2-(4,4,5,5-tetramethyl[1,3,2]-dioxaborolan-2-yl)-1H-indole (1.32 kg, 4.83 mol) dissolved in THF (3.3 L) was added to the reaction over 2-h. The reaction was then stirred at 60° C. for an additional 5 h. The reaction was then allowed to cool to rt. Ethanol (11 L) was added and the suspension was cooled to −10° C. and stirred for at least 1 h. The solids were filtered and washed with ethanol (3×1.5 L). The solid was then suspended in water (22 L) and the pH was adjusted to pH 5-6 using 4M HCl. The resultant suspension was then stirred for a minimum of 12 h and then filtered. The solid was washed with water (3×1.5 L) and ethanol (3×1.5 L) and dried in vacuo to provide trans-4-[4-amino-5-(7-methoxy-1H-indol-2-yl)-imidazo[5,1-f][1,2,4]triazin-7-yl]-cyclohexanecarboxylic acid as a yellow solid (1.00 kg, 77% yield). ¹H NMR (400 MHz, DMSO-d₆): δ 1.51 (dddd, 2H, J=12.0, 12.0, 12.0, 3.2 Hz), 1.74 (dddd, 2H, J=13.2, 13.2, 13.2, 3.6 Hz), 2.03-2.10 (m, 4H), 2.31 (tt, 1H, J=12.4, 3.6 Hz), 3.22 (tt, 1H, J=12.0, 3.2 Hz), 3.93 (s, 3H), 6.65 (d, 1H, J=2.0 Hz), 6.72 (d, 1H, J=7.2 Hz), 6.98 (dd, 1H, J=8.0, 8.0 Hz), 7.18 (d, 1H, J=8.0 Hz), 7.45 (br s, 1H), 7.93 (s, 1H), 11.34 (br s, 1H), 12.08 (br s, 1H).

### Example 9

### trans-4-[4-Amino-5-(7-methoxy-1H-indol-2-yl)-imidazo[5,1-f][1,2,4]triazin-7-yl]-cyclohexanecarboxylate 2-hydroxy-1,1-bis-hydroxymethyl-ethyl-ammonium

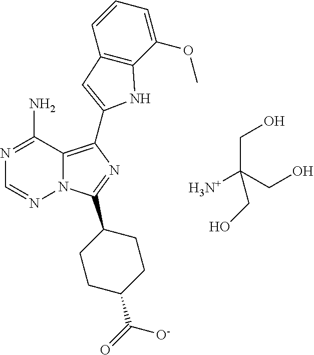

trans-4-[4-Amino-5-(7-methoxy-1H-indol-2-yl)-imidazo[5,1-f][1,2,4]triazin-7-yl]-cyclohexanecarboxylic acid (1.14 kg, 2.81 mol) was suspended in ethanol (11.4 L) and water (11.4 L). Tris(hydroxymethyl)aminomethane (1.03 kg, 8.50 mol) was added and the reaction was heated to reflux. The solution was clarified by hot filtration and then allowed to cool to rt and stirred for 8 h. The suspension was then cooled to −10° C. for 2 h and then filtered. The solid was washed with ethanol (3×2.3 L) and dried in vacuo at 70° C. to provide trans-4-[4-amino-5-(7-methoxy-1H-indol-2-yl)-imidazo[5,1-f][1,2,4]triazin-7-yl]-cyclohexanecarboxylate 2-hydroxy-1,1-bis-hydroxymethyl-ethyl-ammonium as a yellow solid (1.37 kg, 93% yield). ¹H NMR (400 MHz, DMSO-d₆): δ 1.49 (dddd, 2H, J=12.4, 12.4, 12.4, 3.6 Hz), 1.73 (dddd, 2H, J=12.4, 12.4, 12.4, 3.6 Hz), 2.02-2.08 (m, 4H), 2.26 (tt, 1H, J=11.6, 3.2 Hz), 3.20 (tt, 1H, J=12.4, 2.8 Hz), 3.26 (s, 6H), 3.93 (s, 3H), 6.66 (s, 1H), 6.70 (d, 1H, J=7.2 Hz), 6.97 (dd, 1H, J=8.0, 8.0 Hz), 7.17 (d, 1H, J=7.6 Hz), 7.90 (s, 1H).

¹HNMR (400 MHz or 300 MHz) and ¹³C NMR (100.6 MHz) spectra were recorded on Bruker or Varian instruments at ambient temperature with TMS or the residual solvent peak as the internal standard. The line positions or multiples are given in ppm (δ) and the coupling constants (J) are given as absolute values in Hertz (Hz). The multiplicities in ¹H NMR spectra are abbreviated as follows: s (singlet), d (doublet), t (triplet), q (quartet), quint (quintet), m (multiplet), m_{c} (centered multiplet), br or broad (broadened), AA′BB′.

### DEFINITIONS AND ABBREVIATIONS

As used herein, the term “aryl” refers to all-carbon monocyclic, bicyclic, or polycyclic groups of 6 to 12 carbon atoms having a completely conjugated pi-electron system, which may be optionally substituted. Examples of aryl include, but are not limited to, phenyl, 4-chlorophenyl, 4-fluorophenyl, 4-bromophenyl, 3-nitrophenyl, 2-methoxyphenyl, 2-methylphenyl, 3-methyphenyl, 4-methylphenyl, 4-ethylphenyl, 2-methyl-3-methoxyphenyl, 2,4-dibromophenyl, 3,5-difluorophenyl, 3,5-dimethylphenyl, 2,4,6-trichlorophenyl, 4-methoxyphenyl, naphthyl, 2-chloronaphthyl, 2,4-dimethoxyphenyl, 4-(trifluoromethyl)phenyl, and 2-iodo-4-methylphenyl.

The terms “heteroaryl” refer to a substituted or unsubstituted monocyclic, bicyclic, or polycyclic group of 5 to 12 ring atoms containing one or more ring heteroatoms selected from N, O, and S, the remaining ring atoms being C, and, in addition, having a completely conjugated pi-electron system. Examples of such heteroaryl rings include, but are not limited to, furyl, thienyl, pyrrolyl, pyrazolyl, imidazolyl, oxazolyl, isoxazolyl, thiazolyl, isothiazolyl, triazolyl, oxadiazolyl, thiadiazolyl, tetrazolyl, pyridyl, pyridazinyl, pyrimidinyl, pyrazinyl, and triazinyl. The terms “heteroaryl” also include heteroaryl rings with fused carbocyclic ring systems that are partially or fully unsaturated, such as a benzene ring, to form a benzofused heteroaryl. For example, benzimidazole, benzoxazole, benzothiazole, benzofuran, quinoline, isoquinoline, quinoxaline, indole, and the like. Furthermore, the terms “heteroaryl” include fused 5-6, 5-5, 6-6 ring systems, optionally possessing one nitrogen atom at a ring junction. Examples of such heteroaryl rings include, but are not limited to, pyrrolopyrimidinyl, imidazo[1,2-a]pyridinyl, imidazo[2,1-b]thiazolyl, imidazo[4,5-b]pyridine, pyrrolo[2,1-f][1,2,4]triazinyl, and the like. Heteroaryl groups may be attached to other groups through their carbon atoms or the heteroatom(s), if applicable. For example, pyrrole may be connected at the nitrogen atom or at any of the carbon atoms.

The term “C₁-C₁₀ alkyl” includes both branched and straight chain alkyl groups. Typical alkyl groups are methyl, ethyl, n-propyl, isopropyl, n-butyl, sec-butyl, isobutyl, tert-butyl, n-pentyl, isopentyl, n-hexyl, n-heptyl, isooctyl, nonyl, decyl, and the like.

The term “halo” refers to fluoro, chloro, bromo, or iodo.

The term “alkoxy” includes both branched and straight chain terminal alkyl groups attached to a bridging oxygen atom. Typical alkoxy groups include methoxy, ethoxy, n-propoxy, isopropoxy, tert-butoxy and the like.

Unless otherwise specified, the term “C₃-C₁₂ cycloalkyl” refers to a 3-12 carbon mono-cyclic, bicyclic, or polycyclic aliphatic ring structure, optionally substituted with for example, alkyl, hydroxy, oxo, and halo, such as cyclopropyl, methylcyclopropyl, cyclobutyl, cyclopentyl, 2-hydroxycyclopentyl, cyclohexyl, 4-chlorocyclohexyl, cycloheptyl, cyclooctyl, and the like.

The term “purification” in the context of purification of product from a reaction mixture refers to chromatography or recrystallization.

### Abbreviations

| | |
|---|---|
| 1-HOAT | 1-hydroxy-7-azabenzotriazole |
| 1-HOBt | 1-hydroxybenzotriazole hydrate |
| Bn | Benzyl group |
| Boc | tert-butoxycarbonyl |
| BOP-Cl | bis(2-oxo-3-oxazolidinyl)phosphinic chloride |
| br | Broad |
| Cbz | benzyloxycarbonyl |
| CD₃OD | Deuterated methanol |
| CDCl₃ | Deuterated chloroform |
| CDI | 1,1′-carbonyldiimidazole |
| d | Doublet |
| DBN | 1,5-diazabicyclo[4.3.0]non-5-ene |
| DBU | 1,8-diazabicyclo[5.4.0]undec-7-ene |
| DCC | 1,3-dicyclohexylcarbodiimide |
| DCM | dichloromethane |
| DMC | 2-chloro-1,3-dimethylimidazolinium chloride |
| dd | Doublet of doublets |
| DEPC | diethyl cyanophosphonate |
| DIEA | diisopropylethylamine |
| DMF | N,N-dimethylformamide |
| DMSO | dimethyl sulfoxide |
| EDC | 1-(3-dimethylaminopropyl)-3-ethylcarbodiimide hydrochloride |
| EDTA | ethylenediaminetetraacetic acid |
| EGTA | ethyleneglycol-bis(β-aminoethyl)-N,N,N′,N′-tetraacetic Acid |
| ESI | Electrospray Ionization for mass spectrometry |
| Et₃N | triethylamine |
| EtOAc | ethyl acetate |
| EtOH | ethanol |
| Fmoc | Fluorene methyloxycarbonyl |
| HATU | O-(7-azabenzotriazol-1-yl)-N,N,N′,N′-tetramethyluronium |
| | hexafluorophosphate |
| HBTU | O-benzotriazol-1-yl-N,N,N′,N′-tetramethyluronium |
| | hexafluorophosphate |
| HCl | Hydrochloric acid |
| HEPES | 4-(2-hydroxyethyl)-1-Piperazineethane sulfonic acid |
| HOBT | 1-hydroxybenzotriazole |
| HRMS | High Resolution Mass Spectroscopy (electrospray ionization |
| | positive scan) |
| K₃PO₄ | Potassium phosphate |
| LCMS | Liquid Chromatography - Mass Spectroscopy |
| LRMS | Low Resolution Mass Spectroscopy (electrospray or |
| | thermospray ionization positive scan) |
| LRMS | Low Resolution Mass Spectroscopy (electrospray ionization |
| (ES⁻) | negative scan) |
| m | Multiplet |
| m/z | Mass spectrum peak |
| MEM | Minimum essential medium |
| MeOH | methanol |
| MHz | Megahertz |
| MS | Mass spectroscopy |
| NaH | Sodium hydride |
| NMM | N-methylmorpholine |
| NMP | 1-methyl-2-pyrrolidinone |
| NMR | Nuclear Magnetic Resonance |
| PG | Protecting group. Exemplary protecting groups |
| | include Boc, Cbz, Fmoc and benzyl |
| Pg. | Page |
| q | Quartet |
| Rpm | Revolutions per minute |
| s | Singlet |
| t | Triplet |
| TFA | trifluoroacetic acid |
| THF | Tetrahydrofuran |
| TLC | Thin layer chromatography |
| Vol. | Volume |
| δ | Chemical shift |
| DEA | Diethylamine |

1. A process of preparing a compound of Formula (I) or a salt thereof: 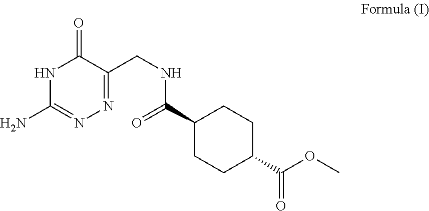
comprising hydrogenating 3-amino-6-((dibenzylamino)methyl)-1,2,4-triazin-5(4H)-one in the presence of a suitable catalyst, a suitable acid, a suitable solvent and at a suitable reaction temperature;
removing the catalyst;
reacting the intermediate product with trans-4-[(2,5-dioxopyrrolidin-1-yl)oxy]carbonylmethylcyclohexanecarboxylate in the presence of a suitable base to provide a compound of Formula (I);
wherein said compound of Formula (I) is obtained on a scale of at least about 1.5 kg.

2. The process according to claim 1 wherein said catalyst of the hydrogenation reaction comprises palladium on carbon.

3. The process according to claim 1 wherein said acid of the hydrogenation reaction comprises acetic acid.

4. The process according to claim 1, wherein said solvent of the hydrogenation reaction comprises at least one of water or acetic acid.

5. The process according to claim 1, wherein said reaction temperature of the hydrogenation reaction is about 30° C. to 90° C.

6. The process according to claim 1, wherein said base comprises triethylamine or sodium carbonate.

7. The process according to claim 1, wherein the overall yield of Formula (I) in the process is at least about 60% at a scale resulting in at least about 1.5 kg.

8. The process according to claim 1, wherein the compound of Formula (I) is further reacted according to reaction Scheme 2: 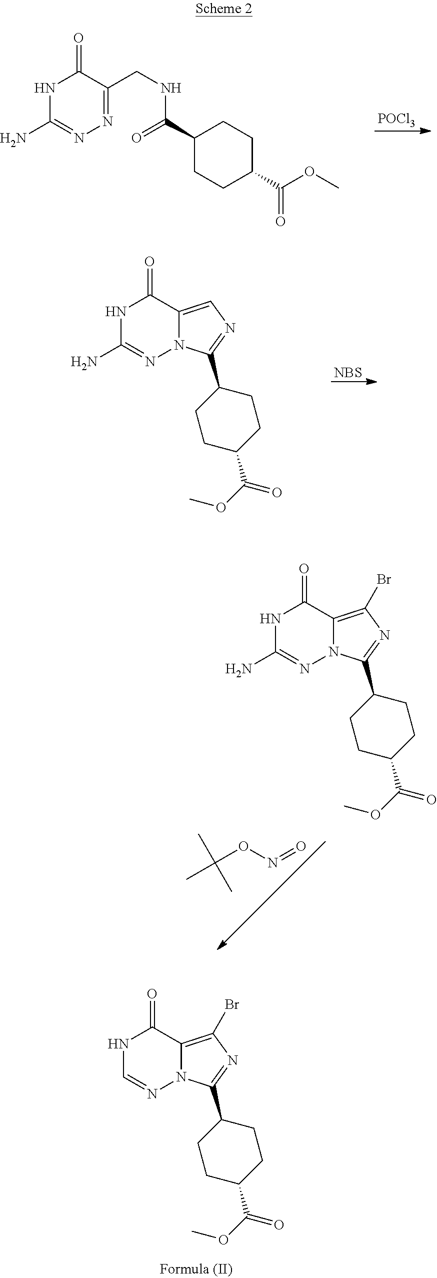
wherein
(A) the compound of Formula (I) is reacted with a suitable amount of POCl₃ in a suitable solvent to provide trans-4-(2-amino-4-oxo-3,4-dihydro-imidazo[5,1-f][1,2,4]triazin-7-yl)-cyclohexanecarboxylic acid methyl ester;
(B) the product from (A) is reacted with N-bromosuccinimide in a suitable solvent at a suitable reaction temperature for a suitable reaction time to provide trans-4-(2-amino-5-bromo-4-oxo-3,4-dihydro-imidazo[5,1-f][1,2,4]triazin-7-yl)-cyclohexanecarboxylic acid methyl ester; and
(C) the product obtained from (B) is reacted with a suitable amount of tert-butyl nitrite in a suitable solvent to provide Formula (II).

9. The process according to claim 8 wherein said amount of POCl₃ in (A) is about 1.3 to about 2 equivalents with respect to Formula (I).

10. The process according to claim 8, wherein said solvent of (A) comprises at least one of acetonitrile, 1,2-diethoxyethane, or dimethoxyethane.

11. The process according to claim 1, wherein the yield of said (A) is at least about 95% and the scale is at least about 1.5 kg of trans-4-(2-amino-4-oxo-3,4-dihydro-imidazo[5,1-f][1,2,4]triazin-7-yl)-cyclohexanecarboxylic acid methyl ester product.

12. The process according to claim 1, wherein said reaction temperature of (B) is about −10° C. to 40° C.

13. The process according to claim 1, wherein the yield of said (B) is at least about 79% and the scale is at least about 2 kg of trans-4-(2-amino-5-bromo-4-oxo-3,4-dihydro-imidazo[5,1-f][1,2,4]triazin-7-yl)-cyclohexanecarboxylic acid methyl ester product.

14. The process according to claim 1, wherein said solvent of (C) comprises at least one of tetrahydrofuran or dimethylformamide.

15. The process according to claim 1, wherein said amount of tert-butyl nitrite in (C) is about 2 to 3 equivalents with respect to trans-4-(2-amino-5-bromo-4-oxo-3,4-dihydro-imidazo[5,1-f][1,2,4]triazin-7-yl)-cyclohexanecarboxylic acid methyl ester.

16. The process according to claim 1, wherein the Formula (II) compound is obtained on a scale of at least about 2 kg at an overall yield of Scheme 2 of at least about 60%.

17. The process according to claim 1, wherein the compound of Formula (II) is further reacted according to reaction Scheme 3: 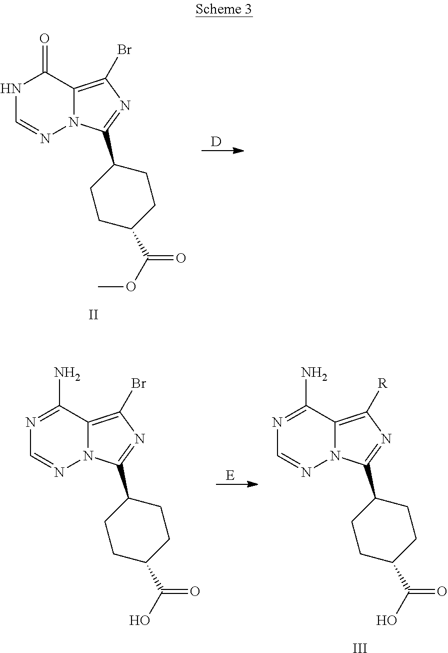
wherein
(D) the compound of Formula (II) is treated with 1,2,4-triazole and POCl₃, the resulting intermediate compound is reacted with ammonia, then hydrolyzed with base to provide trans-4-(4-amino-5-bromo-imidazo[5,1-f][1,2,4]triazin-7-yl)-cyclohexanecarboxylic acid; and
(E) the product from (D) is reacted with an R-boronic acid/ester in the presence of a suitable ligand, a suitable catalyst and a suitable base, and in a suitable solvent to provide a compound of formula (III);
wherein R is an optionally substituted aryl or optionally substituted heteroaryl group.

18. The process according to claim 17, wherein the ammonia is in an alcohol solvent.

19. The process according to claim 17, wherein the R-boronic acid/ester is 7-methoxy-2-(4,4,5,5-tetramethyl[1,3,2]-dioxaborolan-2-yl)-1H-indole.

20. The process according to claim 17, wherein the yield of said (D) is about 90% and the scale is at least about 1.25 kg (D) product.

21. The process according to claim 17, wherein said ligand of (E) is 3,3′,3″-phosphinidynetris(benzenesulfonic acid) trisodium salt, or 4,4′-(phenylphosphinidene)bis-benzensulfonic acid, dipotassium salt hydrate.

22. The process according to claim 17, wherein said catalyst of (E) comprises palladium (II) acetate.

23. The process according to claim 17, wherein said solvent of (E) comprises a mixture of water, ethanol, and tetrahydrofuran.

24. The process according to claim 17, wherein said (E) base comprises Na₂CO₃.

25. The process according to claim 17, wherein:
the R-boronic acid/ester of (E) is 7-methoxy-2-(4,4,5,5-tetramethyl[1,3,2]-dioxaborolan-2-yl)-1H-indole;
the ligand of (E) comprises 3,3′,3″-phosphinidynetris(benzenesulfonic acid) trisodium salt or 4,4′-(phenylphosphinidene)bis-benzensulfonic acid, dipotassium salt hydrate;
the catalyst of (E) comprises palladium (II) acetate; and
the base of (E) comprises Na₂CO₃.

26. The process according to claim 17, wherein the final product is obtained on a scale of at least about 10 kg at an overall yield of Scheme 3 of at least about 72%.

27. The process according to claim 25, wherein the product is further reacted with tris(hydroxymethyl)aminomethane to provide a tromethamine salt.
